(19) United States
(12) Patent Application Publication
(10) Pub. No.: US 2006/0009721 A1
(43) Pub. Date: Jan. 12, 2006

(54) Orthopedic fiberglass bandage with a non-fray substrate

(75) Inventor: John C. Evans, Lancashire (GB)

Correspondence Address: ADAMS EVANS P.A., 2180 TWO WACHOVIA CENTER, CHARLOTTE, NC, 28282, US

(73) Assignee: BSN Medical, Inc., Wilmington, DE (US)

(21) Appl. No.: 10/531,057

(22) PCT Filed: Nov. 25, 2003
(86) PCT No.: PCT/US03/37884

§ 371 Date: Apr. 12, 2005

Related U.S. Application Data
(60) Provisional application No. 60/429,055, filed on Nov. 25, 2002.

(51) Int. Cl. A61F 5/00

(52) U.S. Cl. 602/5; 602/6; 602/8

(57) ABSTRACT

A substrate for a medical bandaging material is a warp knitted fabric in which the chain stitches are constructed from fiberglass yarns and the inlay stitch is constructed from an inelastic low modulus polymeric yarn, such that fraying and unraveling of a cut edge of the substrate is prevented. The medical bandaging product includes the substrate impregnated or coated with a moisture-curable resin and optionally covered with a tubular hydrophobic wrapping. The bandaging product is stored in a moisture-proof container before use.

### TECHNICAL FIELD AND BACKGROUND OF THE INVENTION

This invention relates generally to the field of orthopedic medicine and knitted fiberglass fabrics commonly used in casting tape systems. More particularly, the present invention relates to an improved bandaging product utilizing an extensible knitted fiberglass fabric. A polypropylene textured or intermingled yarn is incorporated into the structure of the fabric for preventing the fabric from unraveling and fraying. Methods for constructing and applying the bandaging product are also disclosed.

Fiberglass casting tapes and medical bandages are commonly used to treat injuries such as broken bones. Such injuries typically require immobilization of a body member. Casting tapes and bandages used to treat these injuries must possess certain characteristics such as porosity, surface area, conformability, elasticity and thickness. Knitted fiberglass fabrics suitable for use in such casting tapes and bandages also preferably have extensibility in the lengthwise and widthwise direction, which permits the casting tape or bandage to be wrapped around body members to repair and support fractures.

The use of high-modulus yarns such as fiberglass as resin reinforcements in bandages or other composite materials is not new. Fiberglass yarns provide strength to cured bandaging products by being woven or knitted into a fabric which is then coated or impregnated with a resin that hardens when cured. Unfortunately, the knitted fiberglass materials presently used in bandaging products also have a tendency to fray and unravel when the materials are cut during cast application. Furthermore, conventional knitted fiberglass material has a tendency to form hard edges and rough surfaces from which wiry, sharp tendrils of fiber protrude after the bandaging product hardens. These tendrils cause discomfort to the patient, even when the affected limb, thumb, skin or other body part is protected by padding.

A number of prior art casting fabrics and bandaging materials exist that unsuccessfully attempt to address the problems caused by frayed edges. The majority of commercially available, resin-coated, fiberglass-knitted casting fabrics utilize either heat-setting or resin binding methods to attempt to control frayed and unraveled edges. However, these methods do not address the problem of frayed and unraveled ends forming along edges of the bandaging material after the material is cut during the cast application process. Once such bandaging material has been applied and hardens, those unraveled yarns evolve into wire-like tendrils that protrude from the cast and create discomfort for the patient. Folding the frayed edges and tucking them under the applied bandage does nothing to prevent the irritating tendrils from forming, but instead increases the likelihood that the wiry fiberglass tendrils will come into contact with the skin of the patient.

Another method for combating frayed and unraveled bandage edges involves the use of a binder resin. The binder resin is applied to the fabric substrate as a thin stretchable coating that bonds to the fibers of the fabric, and is usually applied to the trailing end of the substrate. One prior art use of binder resins involves coating binder material onto standard Raschel knitted fiberglass substrates. In those Raschel-knitted fiberglass substrates which utilize elastic fibers, incorporating the binder resin into the substrate actually increases the amount of fraying and unraveling observed.

This invention overcomes the disadvantages of prior art substrates by providing a medical bandage featuring a resin-impregnated substrate formed from a knitted fiberglass fabric having a structure in which a polypropylene textured or intermingled yarn has been incorporated to prevent the cut ends of the fabric from unraveling and fraying. The polypropylene is incorporated into the inlay stitch, which results in a fiberglass fabric having a softer edge when the finished bandaging product is cut and molded around body member extremities such as the elbows, feet, and especially the thumb. Incorporating polypropylene into the inlay stitch further prevents discomfort to a patient by eliminating the wire-like tendrils that typically result from fiberglass threads created when conventional casting tapes are cut and subsequently undergo curing.

Bandages incorporating the unique fiberglass and polypropylene inlay-stitched material of the present invention do not require the use of binder resin, yet achieve the same strength and quality of those prior art products in which binder resin appears. Because binder resin is not required, the resulting bandages are less expensive to manufacture and purchase than prior art bandages, yet are equally durable in rigidity and strength. In addition, the bandages provide greater safety and comfort to patients. The unique fabric of the present invention provides optimum non-fray performance from a fiberglass bandage material having a much smoother finish, softer edges, and a non glass-like hand.

### SUMMARY OF THE INVENTION

It is therefore an object of the present invention to provide a medical bandaging product with a knitted fiberglass substrate having features that facilitate consistency and conformity in the fiberglass threads when cut, thereby allowing the fiberglass threads to remain in a knotted or looped formation rather than to spring loose.

It is another object of the present invention to provide a medical bandaging product having a substrate that does not fray during manufacture or along cut edges of the bandaging product during the cast application process.

It is another object of the present invention to provide a medical bandaging product that may be cut and applied without the raveled edges, wire-like fiberglass tendrils and associated irritation to the patient commonly exhibited with conventional fiberglass casting tapes and bandages.

It is another object of the present invention to provide a medical bandaging product that includes a knitted substrate incorporating a polypropylene yarn in the inlay stitch. The yarn has a sufficiently low modulus to prevent fraying and unraveling of the cut edges of the bandaging product before, during and after the cast application process, thereby resulting in a cast having a smoother finish.

These and other objects and advantages of the present invention are achieved in the preferred embodiments disclosed below by providing a warp knitted substrate for a medical bandaging material in which the chain stitches are constructed from fiberglass yarns and the inlay stitch is constructed from an inelastic low modulus polymeric yarn, such that fraying and unraveling of a cut edge of the substrate is prevented.

According to another embodiment of the invention, a medical bandaging product is provided including: a warp knitted substrate in which the chain stitches are constructed from fiberglass yarns and the inlay stitch is constructed from an inelastic low modulus polymeric yarn. A reactive system is impregnated into or coated onto the substrate. The reactive system remains stable when maintained in substantially moisture-free conditions and hardens upon exposure to moisture to form a rigid, self supporting structure. A tubular wrapping surrounds the substrate.

According to one preferred embodiment of the invention, the medical bandaging material has an extensibility of between 20 and 35% in the lengthwise direction prior to initiation of the curing process.

According to another preferred embodiment of the present invention, the medical bandage material is formed from a knitted substrate including fiberglass and polypropylene yarns, wherein the fiberglass yarns constitute between 75-95% of the total weight of the substrate.

According to yet another preferred embodiment of the invention, the knitted substrate includes a textured or intermingled polypropylene element which is between 10-25% of the total weight of the substrate.

According to yet another preferred embodiment of the invention, the knitted substrate weighs between 120-170 grams per square meter, but preferably weighs 140 grams per square meter.

According to yet another preferred embodiment of the invention, a medical bandaging product includes: an outer container formed of moisture-impervious material. A medical bandaging material is positioned in the container in substantially moisture-free conditions and sealed therein against entry of moisture until use. The medical bandaging material includes a warp knitted substrate in which the chain stitches are constructed from fiberglass yarns and the inlay stitch is constructed from an inelastic low modulus polymeric yarn. A reactive system is impregnated into or coated onto the substrate, the system remaining stable when maintained in substantially moisture-free conditions and hardening upon exposure to moisture to form a rigid, self supporting structure. A tubular wrapping surrounds the substrate.

According to yet another preferred embodiment of the invention, a method of applying a splint to a selected body part includes providing an initially-moldable, medical bandaging material positioned in a container in substantially moisture-free conditions and sealed therein against entry of moisture until use. The medical bandaging material has a warp knitted substrate in which the chain stitches are constructed from fiberglass yarns and the inlay stitch is constructed from an inelastic low modulus polymeric yarn. A reactive system is impregnated into or coated onto the substrate, the system remaining stable when maintained in substantially moisture-free conditions and hardening upon exposure to moisture to form a rigid, self supporting structure. Optionally, a tubular wrapping covers the substrate. The medical bandaging material is wetted and then urged against a selected body part and into a position whereby the body part is supported in a desired position. The medical bandaging material is molded while flexible to the body part with the body part the desired position. Finally, the medical bandaging material is allowed to harden on the body part.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a perspective view of a medical bandaging product according to one preferred embodiment of the invention;

FIG. 2 is a cut-away fragmentary perspective view of the medical bandaging product shown in FIG. 1;

FIGS. 3 and 4 are stitch diagrams showing the stitch pattern used to form the substrate according to the present invention;

FIGS. 5 is a perspective view of a medical bandaging product according to an alternative embodiment of the invention;

FIG. 6 is a side view of the medical bandaging product shown in FIG. 5 with the clip in a closed position;

FIG. 7 is a perspective view of the medical bandaging product shown in FIGS. 5 and 6 in a dispensing box;

FIG. 8 is a perspective view of the medical bandaging product according to FIG. 5 showing an alternative preferred embodiment of resealing the medical bandaging product;

FIG. 9 is a fragmentary perspective view of one embodiment of the medical bandaging product with a zip end closure;

FIG. 10 is a perspective view of a medical bandaging product according to an alternative embodiment of the invention;

FIG. 11 is a cut-away perspective view of the medical bandaging product shown in FIG. 10; and

FIGS. 12 and 13 illustrate a preferred manner of preparing and applying the medical bandage material according to the present invention.

### DESCRIPTION OF THE PREFERRED EMBODIMENT AND BEST MODE

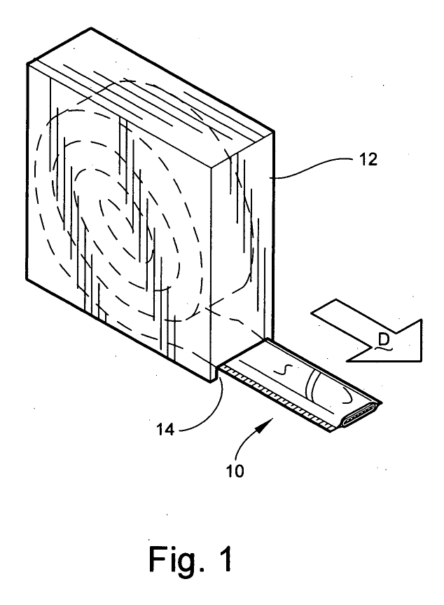

Referring now specifically to the drawings, a medical bandaging product 10 according to a preferred embodiment of the invention is shown generally in FIG. 1. The bandaging product 10 may be sold in any convenient length and is rolled into a coil and positioned within a suitable dispenser 12. While the bandaging product 10 may be sold in any convenient length and be positioned within any suitable dispenser 12, storage container, package or box, a preferred dispenser is one such as that which is illustrated in FIG. 1. The dispenser 12 in FIG. 1 includes a slot 14 defined in a lower corner of the dispenser 12 through which a free end of the bandaging product 10 extends for withdrawing the product 10 from the interior of the dispenser in the direction “D” shown.

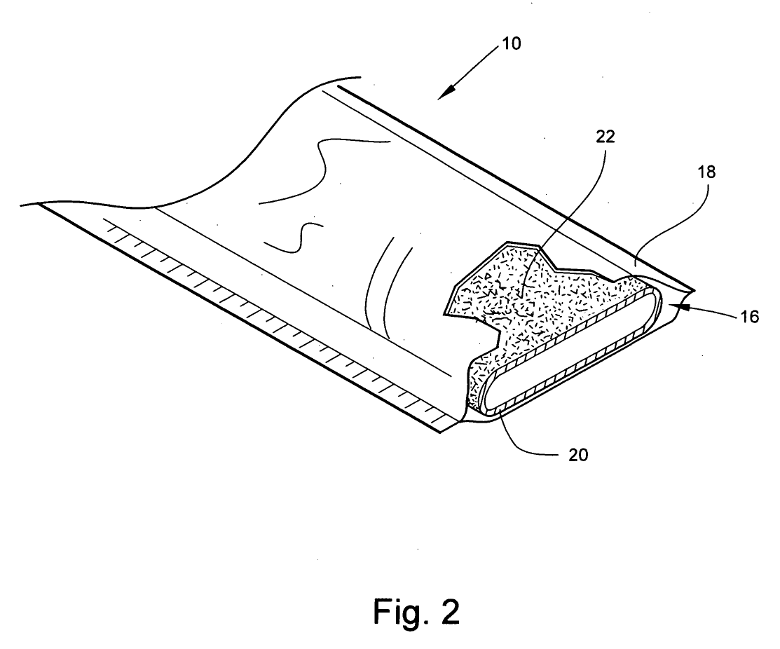

Referring now to FIG. 2, the structure of the bandaging product 10 is shown. The bandaging product 10 includes an elongate medical bandaging material 16 packaged within a moisture-proof foil sleeve 18. Although the sleeve 18 may be formed from any suitable moisture-resistant material, the sleeve 18 is preferably formed from two laminated, elongate foil sheets that have been placed in registration and heat sealed along opposing side edges to form a tube having an open end. Each foil sheet includes an outer, middle and inner layer. The outer layer is preferably formed from a tear-resistant plastic film. The middle layer is preferably formed from aluminum foil and acts as a moisture resistant barrier for protecting the bandage while stored within the package. The inner layer is preferably formed from a plastic film having thermoplastic properties suitable for heat sealing the interior of the package securely against moisture.

As is shown in FIG. 2, the medical bandaging material 16 also includes a substrate 20. In the embodiment shown in FIG. 2, the substrate 20 is covered by and enclosed within a tubular wrapping 22. The tubular wrapping 22 serves as a protective outer cover that enhances patient comfort, and is preferably formed of a soft, flexible, hydrophobic fiber. However, because of the characteristics of the improved substrate 20 of the invention, alternative embodiments of the bandaging material 10 may be formed without the tubular wrapping 22.

The substrate 20 is a knitted material formed from fiberglass and polypropylene yarns, and exhibits an extension (elongation) of approximately 20 to 80% in the widthwise direction and 20 to 100% in the lengthwise direction prior to coating or impregnating the substrate with a curable resin. Preferred ranges of extension are between 30 and 50% in the widthwise direction and 20 to 60% in the lengthwise direction. The substrate 20 may have any suitable thickness and any weight per unit area.

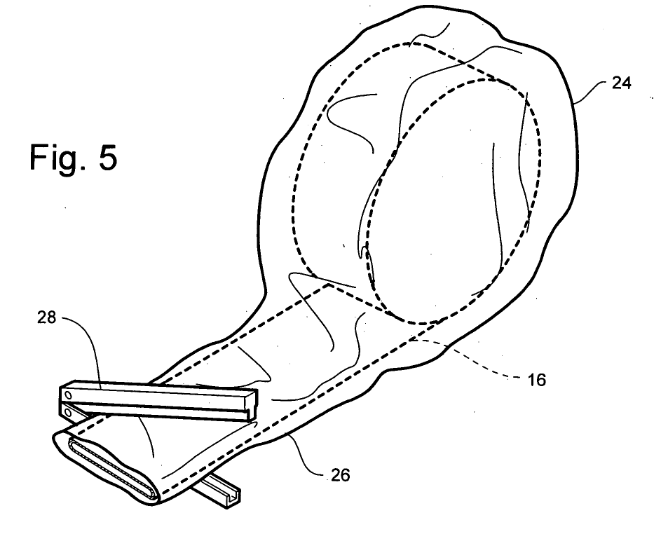

Once the curable resin has been applied to the substrate 20, the substrate 20 exhibits an extension of approximately 10 to 40% in the widthwise direction and 15 to 40% in the lengthwise direction. Optimum conformability of the substrate 20—the ability of the substrate 20 to conform to the shape of the anatomy of the patient—is achieved when the extension is between 10 and 30% in the widthwise direction and 15 to 35% in the lengthwise direction. As is described in greater detail below with reference to FIG. 5, the substrate 20 is a warp-knitted fabric in which the pillar, or chain, stitches are constructed from fiberglass yarns and the inlay stitch is formed using polypropylene yarn. The fiberglass yarns are formed from an “E” glass fiber in a count range of 34 to 136 Tex; however, a preferred count range of the fiberglass yarns utilized in the present invention is 68 Tex. The polypropylene yarn used in the inlay stitch may be flat, intermingled, textured or spun using yarns having a count range of 78 to 220 Decitex, with the preferred count range being 156 Decitex. Although polypropylene yarns are preferred for forming the inlay stitch, other inelastic yarns having a low modulus may also be used. Such yarns include but are not limited to those formed from polyester, nylon and polyethylene.

The density of the threads of the substrate 20 used in fiberglass casting tapes and other medical bandages is critical to successfully achieving optimum non-fray performance. The density of the threads in the substrate 20 of the present invention is preferably between 40 and 60 stitches, or wales, in the widthwise direction and between 70 and 90 stitches, or courses, in the lengthwise direction.

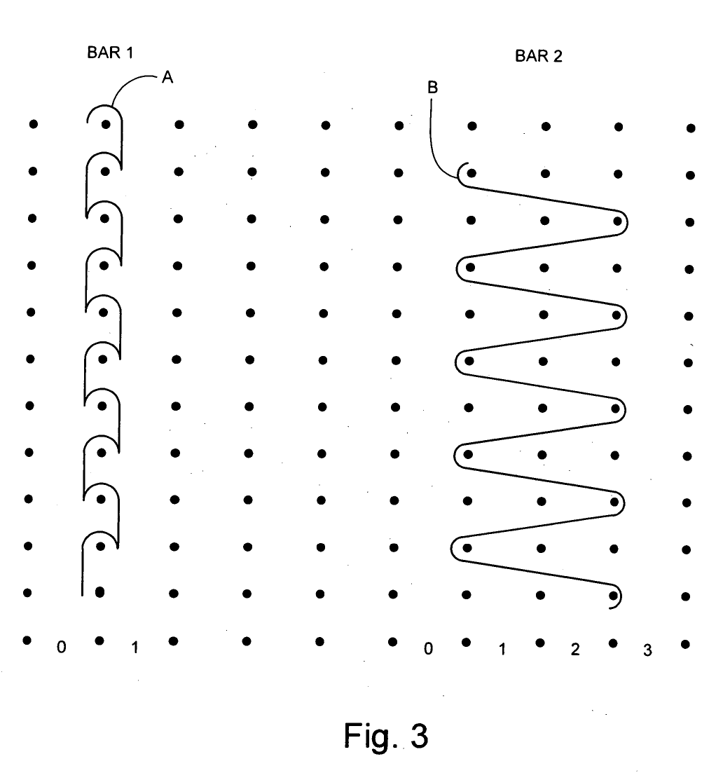

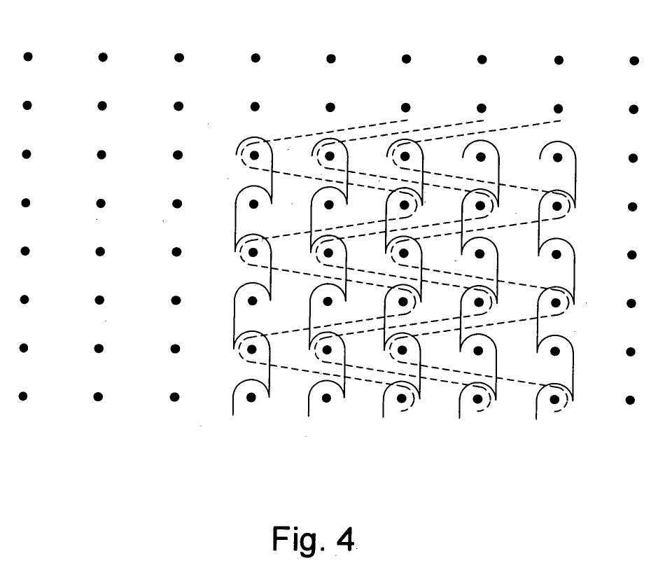

Referring now to FIG. 3, the substrate 20 of the present invention is preferably a warp-knitted substrate, and is knitted on a knitting machine employing two guide bars. The guide bars are shown in the stitch diagram illustrated in FIG. 3 as Bar 1 and Bar 2. Two yarns, yarns A and B, are threaded on Bar 1 and Bar 2, respectively. Yarn A is selected from one of the fiberglass yarns described above. While yarn B is formed from one of the yarns described above, yarn B is preferably formed from polypropylene. Yarn B may alternatively be formed from polyester, nylon or polyethylene fibers, or a mixture of one or more of such fibers. FIG. 4 illustrates a stitch diagram in which all of the guide bars are knitting simultaneously.

After the substrate 20 is knitted, it is coated or impregnated with a suitable resin. The resin may be coated onto the surface of the substrate in any thickness; however, it is imperative that the resin layers remain thin to permit the curing agent will rapidly saturate the resin and activate the curing process. Substrates having a high volume to surface ratio are suited for being coated with a thin resin layer. While any suitable moisture or water-curable resins which will satisfy the functional requirements of an orthopedic cast may be used, the preferred resin is moisture or water-curable and includes a polyurethane prepolymer.

The resin used to coat or impregnate the substrate 20 may also be a moisture-curable resin such as the polyisocyanate resin described in full in the present applicant's U.S. Pat. No. 4,770,299. The resin is synthesized using a reactive system that remains stable when maintained in substantially moisture-free conditions, yet hardens upon exposure to sufficient moisture to form a rigid, self-supporting structure. A typical formation of the reactive system is as follows:

Typical Formulation

| | | |
|---|---|---|
| Isonate 143L or | polyisocyanate | 50.0% |
| Mondur CD or | | |
| Rubinate XI168 | | |
| Pluracol P1010 | polyol | 46.6% |
| DC-200 Silicone | defoamina aaent | 0.30% |
| Benzoyl Chloride | stabilizer | 0.10% |
| Thancat↓ DM-70 | catalyst | 3.0% |
| | | 100% |

A complete discussion of the parameters of the reactive system, the manner of production and the variables which apply are found in U.S. Pat. No. 4,411,262.

The resin remains in a viscous state as long as the resin is not exposed to moisture. This permits the substrate to remain flexible and moldable until the resin is exposed to moisture, and for a short period of time after such exposure occurs. The rate at which the resin cures can be controlled to some extent by the quantity of water to which the resin is exposed. Briefly immersing the resin in water will cause the resin to rapidly cure. In contrast, merely exposing the resin to open air will result in a curing process having a significantly slower reaction rate which will be proportional to the amount of moisture in the air to which the resin is exposed.

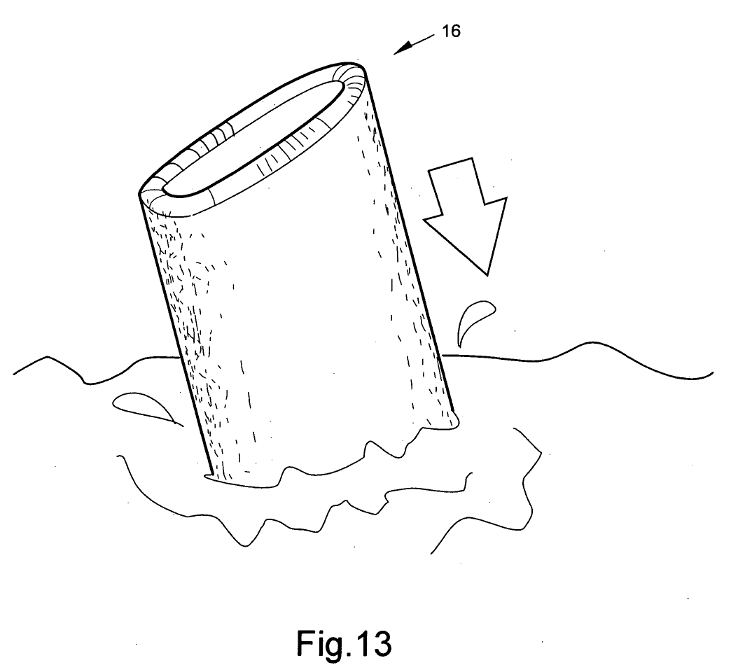

The rate at which the curing agent penetrates the resin on the substrate 20 influences not only the amount of setting time required, but also the rigidity of the resulting cured cast. As described in greater detail below with reference to FIG. 13, the curing agent is preferably water.

Referring now to FIG. 5, an alternative storage package for the medical bandage material of the present invention is illustrated. In particular, a desired length of coiled medical bandage material 16 shown in FIG. 5 is contained within a sealed, moisture-impervious foil bag 24. The coiled medical bandaging material 16 includes the same components and is formed from the same materials as the bandaging material 16 described above with reference to FIGS. 1 through 4. Furthermore, the foil bag 24 is formed from the same laminated foil material as that which forms the sleeve enclosing the medical bandaging material shown in FIGS. 1 and 2.

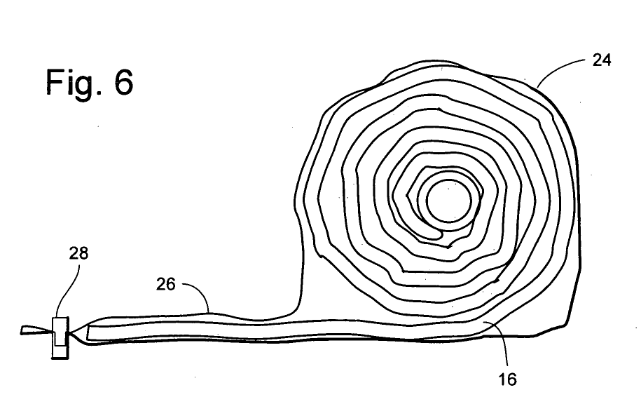

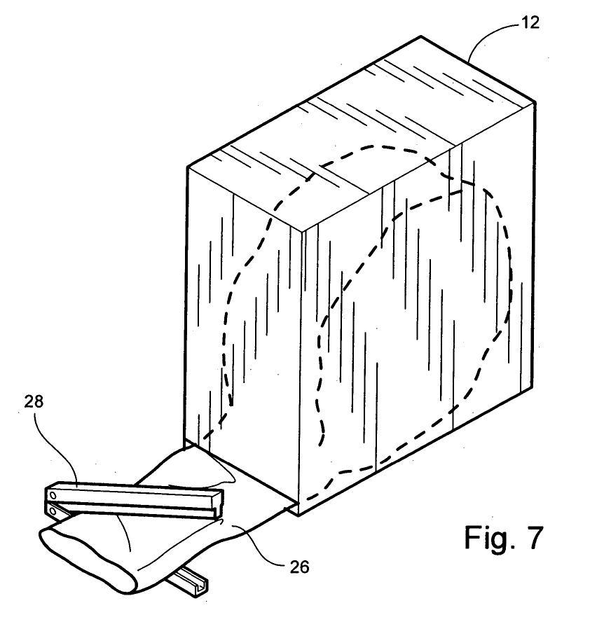

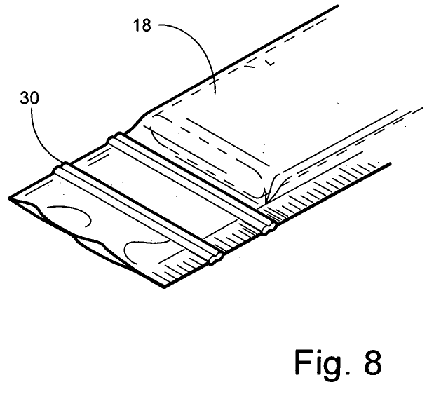

Referring again to FIG. 5, the bag 24 includes an enlarged enclosed area within which the medical bandaging material 16 is positioned. The bag 24 also includes an elongate dispensing sleeve 26 that defines an opening through which a predetermined length of the medical bandaging material 16 may be dispensed. The bag 24 preferably fits relatively snugly around the medical bandaging material to inhibit moisture entry through the opening of the sleeve 26. In order to keep moisture from entering the bag 24 when medical bandaging material 16 is not being dispensed through the opening, sealing means such as the clamps shown in FIGS. 5, 6, and 7, a zip-type closure 30 such as shown in FIG. 8 or a tape strip may be used to close the open end of the sleeve 26. Each of the sealing means shown in FIGS. 5 through 8 may also be used to close the opening of the foil sleeve 18 of the bandaging product 10 described above with reference to FIG. 1.

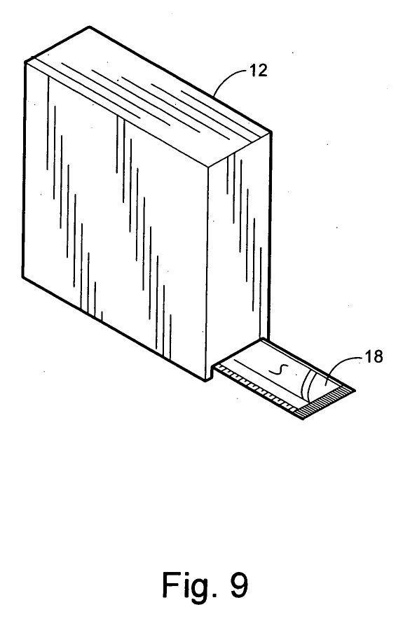

The open end of each of the sleeves in FIGS. 1 and 5 can also be sealed using a clip to hold a folded end of the sleeve closed. A soft, conformable gasketing device can also be used. Such a device would utilize spring loaded compression, leverage clamping or screw action of sufficient strength to keep the sleeve sealed against moisture entry. In addition, a pair of spring loaded rollers can be used. Spring loaded rollers form a better seal by rolling backwards slightly when compressed, which in turn causes the bandaging material to be pushed back slightly into the sleeve. The bandaging material may alternatively be pushed back into the sleeve a sufficient distance (approximately one inch) to allow the open end of the sleeve to be heat sealed in a manner like that which is shown in FIG. 9.

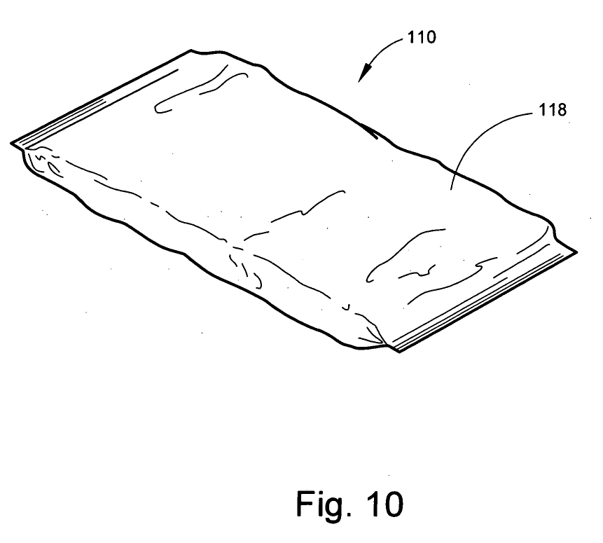

Referring now to FIG. 10, a medical bandaging product 110 according to an alternative embodiment of the invention is shown. The medical bandaging product 110 includes a moisture-impervious package 118 formed from the same foil material used to form the continuous sleeve 18 and bag 24 described above with reference to FIGS. 1 and 5, respectively. In particular, the package shown in FIG. 10 is formed from two laminated sheets that are placed in registration and heat sealed along the edges. The medical bandaging product 110 also includes a medical bandage material 116 that is maintained under moisture-free conditions within the package until the package 118 is opened for use.

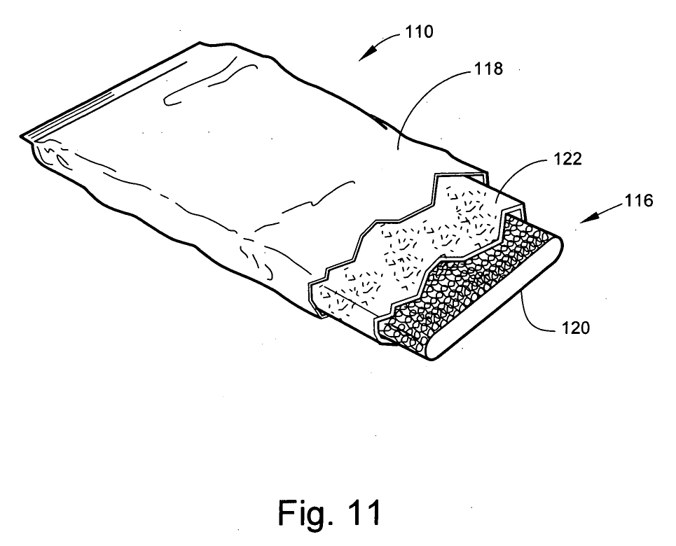

Referring now to FIG. 11, with the exception of the dimensions of the bandage, the medical bandaging material 116 includes the same components and is formed from the same materials as the medical bandaging material 16 described above with reference to FIG. 2. Although the substrate 120 of the bandage shown in FIG. 11 is shown encased within an outer cover 122 formed from a soft, flexible, hydrophobic fiber, because of the unique structure and resulting smooth surface of the substrate 120, the bandage may alternatively be formed without the outer cover. The substrate 120 is formed from the same materials and in the same manner as the substrate described above with reference to FIGS. 1 through 4.

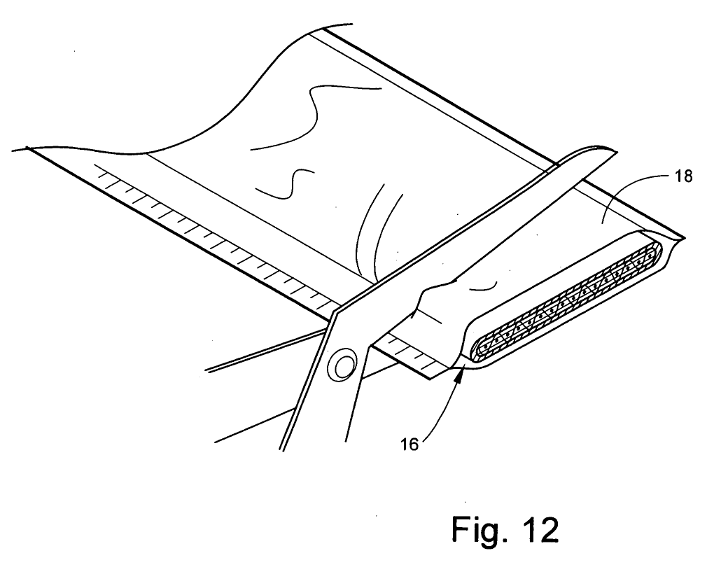

Referring now to FIG. 12, the casting tape of the present invention is used to form an orthopedic cast by first withdrawing the bandaging material 16 through the opening in the dispenser and measuring and cutting a predetermined length of bandaging material using scissors or a knife. The sleeve 18 must be immediately resealed upon cutting the length of bandaging material to ensure that moisture does not enter the bag or coiled sleeve within which the bandaging material is positioned and cause the material to harden.

Referring now to FIG. 13, the curing process is activated by either immersing the bandaging material 16 in or spraying the material with water. Once excess moisture is removed by either squeezing the material 16 or rolling the material 16 in an absorbent towel, the bandage material 16 may be used in any suitable medical procedure to form a cast to immobilize one or more body members. This is achieved by forming an appropriate length of the medical bandaging material 16 to the shape of the body member or members to be immobilized and then overwrapping the moistened medical bandaging material 16 with a conventional elastic bandage and allowing the bandaging material 16 to dry and harden.

Although the medical bandaging product of the present invention may be used to form casts having any suitable number of layers, 2 to 10 layers and 2 to 4 layers are preferred in a non-weight bearing cast. A weight bearing cast preferably has 4 to 10 layers.

A medical bandaging product and material formed of a moisture-curable substrate, and a method of constructing and applying the same is described above. Various details of the invention may be changed without departing from its scope. Furthermore, the foregoing description of the preferred embodiment of the invention and the best mode for practicing the invention are provided for the purpose of illustration only and not for the purpose of limitation.

What is claimed is:

1. A substrate for a medical bandaging material, comprising a warp knitted fabric in which the chain stitches are constructed from fiberglass yarns and the inlay stitch is constructed from an inelastic low modulus polymeric yarn, such that fraying and unraveling of a cut edge of said substrate is prevented.

2. A substrate according to claim 1, wherein said inlay stitch is constructed from polypropylene yarn.

3. A substrate according to claim 1, wherein the fiberglass yarns constitute between 75% and 95% of the total weight of the substrate.

4. A substrate according to claim 1, wherein the polypropylene yarns constitute between 75% and 95% of the total weight of said substrate.

5. A substrate according to claim 1, wherein said fabric weighs between 120-170 grams per square meter.

6. A medical bandaging material, comprising:
(a) a warp knitted substrate in which the chain stitches are constructed from fiberglass yarns and the inlay stitch is constructed from an inelastic low modulus polymeric yarn;
(b) a reactive system impregnated into or coated onto the substrate, the system remaining stable when maintained in substantially moisture-free conditions and hardening upon exposure to moisture to form a rigid, self supporting structure;
(c) a tubular wrapping surrounding the substrate.

7. A medical bandaging according to claim 6, wherein said inlay stitch is constructed from polypropylene yarn.

8. A medical bandaging according to claim 6, wherein said medical bandaging material has an extensibility of between 20% and 35% in the lengthwise direction prior to initiation of the curing process.

9. A medical bandaging according to claim 6, wherein the fiberglass yarns constitute between 75% and 95% of the total weight of the substrate.

10. A medical bandaging according to claim 6, wherein the polypropylene yarns constitute between 75% and 95% of the total weight of the substrate.

11. A medical bandaging according to claim 6, wherein the substrate weighs between 120-170 grams per square meter.

12. A medical bandaging product according to claim 6, wherein said tubular wrapping is formed of a synthetic, hydrophobic fabric.

13. A medical bandaging according to according to claim 6, wherein the reactive system comprises a blended polyisocyanate, polyol, catalyst and stabilizer.

14. A medical bandaging product, comprising:
(a) an outer container formed of moisture-impervious material;
(b) a medical bandaging material positioned in the container in substantially moisture-free conditions and sealed therein against entry of moisture until use, and comprising:
(i) a warp knitted substrate in which the chain stitches are constructed from fiberglass yarns and the inlay stitch is constructed from an inelastic low modulus polymeric yarn;
(ii) a reactive system impregnated into or coated onto the substrate, the system remaining stable when maintained in substantially moisture-free conditions and hardening upon exposure to moisture to form a rigid, self supporting structure;
(iii) a tubular wrapping surrounding the substrate.

15. A medical bandaging product according to claim 14, wherein said inlay stitch is constructed from polypropylene yarn.

16. A medical bandaging product according to claim 14, wherein said medical bandaging material has an extensibility of between 20% and 35% in the lengthwise direction prior to initiation of the curing process.

17. A medical bandaging product according to claim 14, wherein the fiberglass yarns constitute between 75% and 95% of the total weight of the substrate.

18. A medical bandaging product according to claim 14, wherein the polypropylene yarns constitute between 75% and 95% of the total weight of the substrate.

19. The medical bandaging product of claim 14, wherein the substrate weighs between 120-170 grams per square meter.

20. A medical bandaging product according to claim 14, wherein the container is fabricated of an aluminum foil laminate having an outer tear resistant layer, a central aluminum foil layer and an inner heat sealable plastic layer.

21. A medical bandaging product according to claim 14, wherein said tubular wrapping is formed of a synthetic, hydrophobic fabric.

22. A medical bandaging product according to claim 14, wherein the reactive system comprises a blended polyisocyanate, polyol, catalyst and stabilizer.

23. A medical bandaging product according to claim 20, wherein said outer container defines a bag which receives a coil of said medical bandaging material, and an elongated sleeve for dispensing said medical bandaging material.

24. A medical bandaging material according to claim 20 further comprising means for resealing an end of said outer container against the entry of moisture therein after a length of the medical banding product has been removed therefrom.

25. A medical bandaging product according to claim 20 wherein said outer container and said medical bandaging material contained therein are pre cut to a selected length and the ends of said outer container are sealed against the entry of moisture therein.

26. A method of applying a splint to a selected body part, comprising the steps of:
(a) providing an initially-moldable, medical bandaging material positioned in a container in substantially moisture-free conditions and sealed therein against entry of moisture until use, said medical bandaging material comprising:
(I) a warp knitted substrate in which the chain stitches are constructed from fiberglass yarns and the inlay stitch is constructed from an inelastic low modulus polymeric yarn;
(ii) a reactive system impregnated into or coated onto the substrate, the system remaining stable when maintained in substantially moisture-free conditions and hardening upon exposure to moisture to form a rigid, self supporting structure; and
(iii) a tubular wrapping covering the substrate;
(b) wetting said medical bandaging material;
(c) urging said medical bandaging material against said selected body part and into a position whereby the body part is supported in a desired position;
(d) molding the medical bandaging material while flexible to the body part with the body part the desired position; and
(e) allowing the medical bandaging material to harden on the body part.
